(19) United States
(12) Patent Application Publication
(10) Pub. No.: US 2017/0166969 A1
(43) Pub. Date: Jun. 15, 2017

(54) METHOD FOR DETERMINING A SUPPLEMENT COMPOSITION FOR PREVENTING DEVELOPMENT OF DRY INTERMEDIATE AGE-RELATED MACULAR DEGENERATION

(71) Applicant: ArcticDx, Inc., Toronto (CA)

(72) Inventors: Rafall Kustra, Etobicoke (CA); Brent Zanke, Toronto (CA)

(21) Appl. No.: 15/372,966

(22) Filed: Dec. 8, 2016

(30) Foreign Application Priority Data
Dec. 10, 2015 (CA) 2914722

(51) Int. Cl. C12Q 1/68

(52) U.S. Cl. CPC C12Q 1/6883; C12Q 2600/106; C12Q 2600/156; C12Q 2600/16

(57) ABSTRACT

Disclosed is a method for determining a supplement regime for a subject with normal eyes or AREDS category 1 AMD to minimize the development of intermediate dry (AREDS category 3) AMD. The method involves determining the subject's genetic risk profile for the complement factor H gene and ARMS2 gene and administering an antioxidant/carotenoid supplement if the individual has high genetic risk and avoiding the administration of such a supplement, which increases the risk for AMD, in those with low risk disease.

### FIELD OF THE INVENTION

The present invention relates to the fields age-related macular degeneration (AMD) predictive testing and therapeutics. In particular, the invention relates to a method for determining a nutritional supplement regime in a subject to prevent the development of AMD.

### BACKGROUND OF THE INVENTION

Age-related macular degeneration (AMD) causes progressive impairment of central vision and is the leading cause of irreversible vision loss in older Americans (Swaroop A et al., 2007, Hum Mol Genet 16 Spec 2:R174-82). The central geographic atrophy form of AMD is also referred to as dry AMD. Some subjects with dry AMD will have the disease progress into neovascular or exudative AMD, which usually results in blindness. The neovascular or exudative form of AMD is also referred to as wet AMD.

Although the etiology of AMD remains largely unknown, implicated risk factors include age, ethnicity, smoking, hypertension, obesity and diet (Ambati J et al., 2003, Sury Opthalmol 48(3):257-93). Familial aggregation (Klaver C C et al., 1998, Arch Opthalmol 116(5):653-8), twin studies (Hammond C J et al., 2002, Opthalmology 109(4):730-6), and segregation analysis (Heiba I Metal., 1994, 11(1):51-67) suggest that there is also a significant genetic contribution to the disease. The candidate gene approach and genome-wide association studies have consistently implicated the complement factor H (CFH), third component of complement (C3) and second component of complement/factor B (C2/BF) genes, all members of the complement-mediated inflammatory cascade, as well as Age-Related Maculopathy Susceptibility 2 (ARMS2), a gene likely involved in mitochondria-associated pathways.

Much progress has been made in identifying and characterizing the genetic basis of AMD. In a remarkable example of the convergence of methods for disease gene discovery, multiple independent research efforts identified the Y402H variant in the complement factor H (CFH [(MIM 134370]) gene on chromosome 1q32 as the first major AMD susceptibility allele (Haines J L et al., 2005, Science 308(5720):419-21; Hageman G S et al., 2005, Proc Natl Acad Sci USA 102(20):7227-32; Klein R J et al., 2005, Science 308(5720):385-9; Edwards A O et al., 2005, Science 308(5720):421-4; Zareparsi S et al., 2005, Am J Hum Genet 77(1):149-53; Jakobsdottir J et al., 2005, Am J Hum Genet 77(3):389-407). While one of the studies was able to pinpoint CFH on the basis of a whole-genome association study (Klein R J et al., supra), most studies focused on the 1q32 region because it had consistently been implicated by several whole-genome linkage scans. More recently, disease associated haplotypes within the CFH gene have also been shown to be associated with AMD (Li M et al., 2006, Nat Genet 38(9):1049-54). A second genomic region with similarly consistent linkage evidence is chromosome 10q26, which was identified as the single most promising region by a recent meta-analysis of published linkage screens (Fisher S A et al., 2005, Hum Mol Genet 14(15):2257-64).

The Age-Related Eye Disease Study (AREDS), sponsored and conducted by the National Eye Institute (US), provided descriptive data on the clinical course of AMD and attempted to identify factors that influence the development of early disease and progression and evaluated the potential efficacy of high-dose vitamins and minerals to arrest or retarding disease progression. It was a long-term multicenter, prospective study of 4757 persons age 55 to 80 years that assessed the clinical course, prognosis, and risk factors of AMD.

The study was designed to document the clinical course of AMD and determine progression risk determinants through the collection of data on possible risk factors, changes in visual acuity, photographically documenting changes in the macula and self-reported visual function were recorded at regular intervals. A grading system was developed for each of the lesions of AMD. The major AMD outcomes in this study were the development of neovascular disease or the development of geographic atrophy that involves the center of the macula. Eyes developing either of these conditions were considered to have progressed to advanced AMD. Early lesions of AMD, particularly drusen (size, type, and extent) and RPE abnormalities (detachment, atrophy, and pigment disturbances) were graded individually for each study eye.

At the time of the AREDS study design there was evidence for the beneficial effect of antioxidants for AMD. The study was designed to determine if zinc, alone or in combination with a specific vitamin/antioxidant formulation could slow the progression of AMD. Formulations that included zinc included copper to prevent zinc-induced copper-deficiency anemia. Study participants at risk of vision loss with early AMD received combinations of the zinc formulation and the vitamin/antioxidant formulation. Participants without drusen or RPE changes were never assigned to the zinc formulation. Remaining participants (3640) were enrolled in a 2×2 factorial design of antioxidants and zinc.

The results of the AREDS Study was reported in 2001 (see AREDS report no. 8, Arch Ophthalmol 119:1417-36, 2001, which is incorporated herein in its entirety). The average follow-up of the 3640 enrolled study participants, aged 55-80 years, was 6.3 years, with 2.4% lost to follow-up. Comparison with placebo demonstrated a statistically significant odds reduction for the development of advanced AMD with antioxidants plus zinc (odds ratio [OR], 0.72; 99% confidence interval [CI], 0.52-0.98). The ORs for zinc alone and antioxidants alone are 0.75 (99% CI, 0.55-1.03) and 0.80 (99% CI, 0.59-1.09), respectively. Participants with extensive small drusen, nonextensive intermediate size drusen, or pigment abnormalities had only a 1.3% 5-year probability of progression to advanced AMD. Odds reduction estimates increased when these 1063 participants were excluded (antioxidants plus zinc: OR, 0.66; 99% CI, 0.47-0.91; zinc: OR, 0.71; 99% CI, 0.52-0.99; antioxidants: OR, 0.76; 99% CI, 0.55-1.05). Both zinc and antioxidants plus zinc significantly reduced the odds of developing advanced AMD in this higher-risk group. The only statistically significant reduction in rates of at least moderate visual acuity loss occurred in persons assigned to receive antioxidants plus zinc (OR, 0.73; 99% CI, 0.54-0.99). No statistically significant serious adverse effect was associated with any of the formulations.

As a result of the findings of AREDS study, subjects or patients that present with the symptoms of dry AMD are routinely prescribed zinc and antioxidant containing vitamins to delay or prevent the onset of wet AMD. The genetic profile of the subject has not been a consideration when prescribing such course of treatment.

Individuals with no evidence of retinal abnormality were assigned to either placebo or antioxidant treatment only. The study investigators excluded this group from zinc exposure to prevent potential toxic consequences in recognition of their low risk of progression to advanced disease.

### SUMMARY OF THE INVENTION

According to an aspect of the present invention there is provided a method for determining a supplement regime for a subject to prevent the development of age-related macular degeneration (AMD). The method involves determining the subject's risk of developing AREDS category 3 AMD (dry AMD) in a sample from the subject based on their genetic profile for the complement factor H gene; and administering a supplement based on their genetic profile for the complement factor H gene and the Age Related Maculopathy Sensitivity 2 (ARMS2) gene. When the subject has 3 or 4 risk alleles at these 2 sites treatment with a combination of beta carotene, vitamin E and vitamin C is beneficial. When the subject has 0 or 1 risk alleles at these 2 sites treatment with a combination of beta carotene, vitamin E and vitamin C increases the risk of progression to AREDS category 3 age related macular degeneration.

In one embodiment, the subject is without any evidence of age-related macular degeneration.

In another embodiment, the subject's risk of developing AMD-related drusen is determined by analysing the single nucleotide polymorphisms: rs3766405 and rs412852 in the CFH gene.

In a further embodiment, the subject's risk of developing AMD-related drusen is determined by analysing the single nucleotide polymorphisms: rs1048663, rs3766405, rs412852, rs11582939 and/or rs1280514 in the CFH gene.

In a still further embodiment, the subject's risk of developing AMD-related drusen is determined by analysing the single nucleotide polymorphism rs1061170, where an individual is at high risk of developing AMD-related drusen when they are homozygous for the C allele, at medium risk when they are heterozygous for the C allele and at low risk when they are homozygous for the T allele.

In yet a further embodiment, the supplement is a multi-vitamin supplement.

In another embodiment, when the subject is at high risk of developing AMD-related drusen based on their genetic profile for the complement factor H gene the supplement is included the antioxidants vitamin E, vitamin C and carotenoids, such as beta-carotene. In addition, when the subject is at low risk of developing neovascular or exudative AMD based on their genetic profile for the complement factor H (CFH) gene the supplement is free of antioxidants. Carotenoids belong to the category of tetraterpenoids (i.e., they contain 40 carbon atoms, being built from four terpene units each containing 10 carbon atoms). Structurally, carotenoids take the form of a polyene hydrocarbon chain which is sometimes terminated by rings, and may or may not have additional oxygen atoms attached. Carotenoids with molecules containing oxygen, such as lutein and zeaxanthin are known as xanthophylls. The unoxygenated (oxygen free) carotenoids such as α-carotene, β-carotene, and lycopene, are known as carotenes. Carotenes typically contain only carbon and hydrogen (i.e., are hydrocarbons), and are in the subclass of unsaturated hydrocarbons.

In an embodiment, the subject is considered at high risk of developing AMD-related drusen when the subject is homozygous for the C allele at rs3766405 and is homozygous for the C allele at rs412852. The subject is considered at medium risk of developing AMD-related drusen when the subject is heterozygous for the C allele at rs3766405 and is heterozygous for the C allele at rs412852; or homozygous for the C allele at rs3766405 and heterozygous for the T allele at rs412852. The subject is considered at low risk of developing AMD-related drusen when the subject is homozygous for the C allele at rs3766405 and is homozygous for the T allele at rs412852, heterozygous for the C allele at rs3766405 and is homozygous for the C allele at rs412852, heterozygous for the C allele at rs3766405 and is homozygous for the T allele at rs412852, homozygous for the T allele at rs3766405 and is homozygous for the C allele at rs412852, homozygous for the T allele at rs3766405 and is heterozygous for the C allele at rs412852 or homozygous for the T allele at rs3766405 and is homozygous for the T allele at rs412852.

In an embodiment, the subject is considered at risk all for of developing AMD-related drusen when the T allele at rs10490924 (ARMS2 locus) is present, or when this gene has an insertion/deletion polymorphism defined as ARMS2 (NM_001099667.1:c.* 372_815del443ins54). Two copies of the T alleles or two copies of the ARMS2 (NM_001099667.1:c.* 372_815del443ins54) would be considered to constitute 2 AMD risk alleles at this locus.

According to the method described above, the subject's genetic profile can be detected by hybridization, chemical cleavage, direct DNA sequencing, use of restriction enzymes or Southern blotting.

### BRIEF DESCRIPTION OF THE DRAWINGS

FIG. 1 is a Kaplan Meier curve representing the progression from category 1 AMD to category 3 AMD of eyes in antioxidant or placebo-treated subjects having no CFH risk alleles (Risk Group 0), 1 CFH risk allele (Risk Group 1) or 2 CFH risk alleles (Risk group 2) (B). The other eye of patients with these index eyes can be category 1-4.

FIG. 2 is a Kaplan Meier curve representing the progression from category 1 AMD to category 3 AMD of eyes in antioxidant or placebo-treated subjects having no CFH or ARMS2 risk alleles (no risk), just 1 CFH or ARMS2 risk allele (low risk), 2 CFH or ARMS2 risk alleles (medium risk) or 3 or more CFH and ARMS2 risk alleles (high risk). Other eye can be category 1-4. (4 panels: A—no risk alleles, B—1 risk allele, C—2 risk alleles, D—3 or more risk alleles).

FIG. 3 is a Kaplan Meier curve representing the progression of eyes from category 1 AMD (normal eye) to category 3 AMD (dry intermediate AMD) in antioxidant or placebo-treated subjects having no CFH risk alleles (A) or at least 1 risk allele (B). In these subjects the other eye is normal (AREDS category 1). (2 panels: A—0 CFH risk alleles AO- or placebo treated, B—at least 1 CFH risk allele, AO or placebo treated).

FIG. 4 is a Kaplan Meier curve representing the progression from category 1 AMD to category 3 AMD of eyes in antioxidant or placebo-treated subjects having no CFH or ARMS2 risk alleles (no risk), just 1 CFH or ARMS2 risk allele (low risk), 2 CFH or ARMS2 risk alleles (medium risk) or 3 or more CFH and ARMS2 risk alleles (high risk). Other eye has AREDS category 1. (4 panels: A—no risk alleles, B—1 risk allele, C—2 risk alleles, D—3 or more risk alleles).

FIG. 5 is a Kaplan Meier curve representing the progression from category 2 AMD to category 3 AMD of eyes in antioxidant or placebo-treated subjects having no CFH risk alleles (A) or at least 1 risk allele (B). Other eye can be category 1-4. Index eyes are classified by the number of CFH risk alleles within the study subject: No CFH risk alleles (Risk Group 0), 1 CFH risk allele (Risk Group 1) or 2 CFH risk alleles (Risk group 2).

FIG. 6 is a Kaplan Meier curve representing the progression from category 2 AMD to category 3 AMD of eyes in antioxidant or placebo-treated subjects having no CFH or ARMS2 risk alleles (no risk), just 1 CFH or ARMS2 risk allele (low risk), 2 CFH or ARMS2 risk alleles (medium risk) or 3 or more CFH and ARMS2 risk alleles (high risk). Other eye can be category 1-4. (4 panels: A—no risk alleles, B—1 risk allele, C—2 risk alleles, D—3 or more risk alleles).

FIG. 7 is a Kaplan Meier curve representing the progression from category 2 AMD to category 3 AMD of eyes in antioxidant or placebo-treated subjects. Index eyes are classified by the number of CFH risk alleles within the study subject: No CFH risk alleles (Risk Group 0), 1 CFH risk allele (Risk Group 1) or 2 CFH risk alleles (Risk group 2). (B). Other eye has AREDS category 1.

FIG. 8 is a Kaplan Meier curve representing the progression from category 2 AMD to category 3 AMD of eyes in antioxidant or placebo-treated subjects having no CFH or ARMS2 risk alleles (no risk), just 1 CFH or ARMS2 risk allele (low risk), 2 CFH or ARMS2 risk alleles (medium risk) or 3 or more CFH and ARMS2 risk alleles (high risk). Other eye has AREDS category 1. (4 panels: A—no risk alleles, B—1 risk allele, C—2 risk alleles, D—3 or more risk alleles).

### DESCRIPTION OF THE INVENTION

The following description is of an illustrative embodiment by way of example only and without limitation to the combination of features necessary for carrying the invention into effect.

The method described herein is purposed to determine a supplement regime for a subject that has normal eyes or has been previously diagnosed with early stage AMD, corresponding to category 2 of the Age-Related Eye Diseases Study which specifies individuals who have retinal drusen of size less than 125 μM. For the purposes of this disclosure, the term “early dry AMD” will be used hereinafter to describe individuals with AREDS category 2 disease. Individuals with AREDS category 3 disease (drusen>125 μM in size) will be referred to as “intermediate dry AMD”. The neovascular or exudative form of AMD is also referred to as wet AMD. For the purposes of this disclosure, the term “wet AMD” will be used hereinafter to describe the neovascular or exudative form of AMD.

The method described herein analyses the genetic profile of the subject to determine whether a supplement regime containing antioxidants and carotinoids should be administered to a subject with normal eyes or early dry AMD to prevent progression to intermediate dry AMD, or whether such supplements should be avoided. For example, a subject having normal eyes or early dry AMD and a genetic profile that excludes risk alleles at the ARMS2 and CFH gene loci, alone or in combination, should avoid administration of dietary supplements containing the antioxidants vitamin C, vitamin E and the carotenoid β-carotene which accelerate progression of to intermediate dry AMD. Subjects at high genetic risk, defined as the presence of 3 or 4 risk alleles at the CFH and ARMS2 loci should take vitamin E, vitamin C and β-carotene individually or in combination to delay or prevent the onset of intermediate dry AMD.

The supplement regime described herein can be in the form of a single multiple vitamin formulation, or can be a series of individual vitamin formulations, or combinations thereof. In either case, the formulation should be controllable so that antioxidants can be provided or removed depending on the specific needs of the patient.

Antioxidants and the dosages thereof that provide health benefits are known to those skilled in the art, and often include, but are not limited to, vitamins C and E, selenium, and carotenoids, such as beta-carotene, lycopene, lutein, and zeaxanthin. For the purposes of the present discussion antioxidants can be synthetic or be derived from natural sources.

The risk of a subject developing age-related macular degeneration is determined by the genetic profile of the subject, specifically at the genetic loci CFH (Entrez Gene: 3075 Ensembl: ENSG00000000971) and ARMS2 (Entrez Gene: 387715 Ensembl: ENSG00000254636). In particular, single nucleotide polymorphisms (SNPs) in the complement factor H (CFH) gene located on chromosome 1 of the human genome are used to determine the risk of the subject developing wet AMD. Several SNPs in the CFH gene have been shown to be predictors of AMD development and/or predictors of disease progression from dry AMD to wet AMD (Li M et al., Nature Genetics 38(9):1049-1054, 2006, the contents of which are incorporated herein). These SNPs include: rs1048663, rs3766405, rs412852, rs11582939 and rs1280514. In the method described herein, at least rs3766405 and rs412852 are used to determine the risk of a subject with dry AMD developing wet AMD. Since each individual will have two copies of each allele, possible allele combinations at the rs3766405 and rs412852 SNPs include: cytosine (C)/C; C/thymine (T); and T/T. As shown in Table 11, risk of an individual with dry AMD developing wet AMD is determined based on the genotype of the individual at rs3766405 and rs412852.

TABLE 11

| rs3766405 | rs412852 | Risk |
|---|---|---|
| CC | CC | High |
| CT | CT | Medium |
| CC | CT | |
| CC | TT | |
| CT | CC | Low |
| CT | TT | |
| TT | CC | |
| TT | CT | |
| TT | TT | |

In another embodiment, rs1061170 is used to determine risk of a subject with dry AMD developing wet AMD. Since each individual will have two copies of each allele, possible allele combinations at the rs1061170 SNP include: C/C (high risk); C/T (medium risk); and T/T (low risk).

Numerous methods exist for the measurement of a specific polymorphism or SNP. Individuals carrying polymorphisms at one or more markers in the CFH gene may be detected at the DNA level by a variety of techniques. Nucleic acids for diagnosis may be obtained from a patient's cells, such as from blood, urine, saliva, tissue biopsy and autopsy material. The nucleic acid sample can be isolated from a biological sample using standard techniques. The nucleic acid sample may be isolated from the subject and then directly utilized in a method for determining the presence of a polymorphic variant, or alternatively, the sample may be isolated and then stored (e.g., frozen) for a period of time before being subjected to analysis.

Genomic DNA may be used directly for detection or may be amplified enzymatically by using PCR prior to analysis (Saiki R K et al., 1986, Nature 324(6093):163-6). As an example, PCR primers complementary to the nucleic acid of one or more polymorphic variants of the present invention can be used to identify and analyze the presence or absence of the polymorphic variant.

Sequence differences between a reference gene and genes having a polymorphism also may be revealed by direct DNA sequencing. In addition, cloned DNA segments may be employed as probes to detect specific DNA segments. The sensitivity of such methods can be greatly enhanced by appropriate use of PCR or another amplification method. For example, a sequencing primer is used with a double-stranded PCR product or a single-stranded template molecule generated by a modified PCR technique. The sequence determination is performed by conventional procedures with radiolabeled nucleotide or by automatic sequencing procedures with fluorescent-tags.

Genetic testing based on DNA sequence differences may be achieved by detection of alteration in electrophoretic mobility of DNA fragments in gels, with or without denaturing agents. Small sequence deletions and insertions can be visualized by high resolution gel electrophoresis. DNA fragments of different sequences may be distinguished on denaturing formamide gradient gels in which the mobilities of different DNA fragments are retarded in the gel at different positions according to their specific melting or partial melting temperatures (Myers R M et al., 1985, Science 230(4731):1242-6).

Sequence changes at specific locations also may be revealed by nuclease protection assays, such as RNase and 51 protection or the chemical cleavage method (Cotton R G et al., 1988, Proc Natl Acad Sci USA 85(12):4397-401).

Thus, the detection of a specific DNA sequence may be achieved by methods which include, but are not limited to, hybridization, chemical cleavage, direct DNA sequencing or the use of restriction enzymes, (e.g., restriction fragment length polymorphisms (“RFLP”)) and Southern blotting of genomic DNA. In addition, RNA or mRNA expression levels may be specifically determined by a number of different methods, including, but not limited to nuclease protection assay, Northern blot analysis, in situ hybridization or reverse-transcriptase polymerase chain reaction.

In addition to more conventional gel-electrophoresis and DNA sequencing, mutations also can be detected by in situ analysis.

Furthermore, the presence or absence of the polymorphism can be determined using one or both chromosomal complements represented in the nucleic acid sample. Determining the presence or absence of a polymorphic variant in both chromosomal complements represented in a nucleic acid sample is useful for determining the zygosity of an individual for the polymorphic variant (i.e., whether the individual is homozygous or heterozygous for the polymorphic variant). Any oligonucleotide-based diagnostic may be utilized to determine whether a sample includes the presence or absence of a polymorphic variant in a sample. For example, primer extension methods, ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), microarray sequence determination methods, restriction fragment length polymorphism (RFLP), single strand conformation polymorphism detection (SSCP) (e.g., U.S. Pat. Nos. 5,891,625 and 6,013,499), PCR-based assays (e.g., TAQMAN™ PCR System (Applied Biosystems)), and nucleotide sequencing methods may be used.

Oligonucleotide extension methods typically involve providing a pair of oligonucleotide primers in a polymerase chain reaction (PCR) or in other nucleic acid amplification methods for the purpose of amplifying a region from the nucleic acid sample that comprises the polymorphic variation. One oligonucleotide primer is complementary to a region 3′ or downstream of the polymorphism and the other is complementary to a region 5′ or upstream of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GENEAMP™, systems available from Applied Biosystems. Also, those of ordinary skill in the art will be able to design oligonucleotide primers based upon the nucleotide sequences set forth in SEQ ID NOs:1-3.

Also provided is an extension oligonucleotide that hybridizes to the amplified fragment adjacent to the polymorphic variation. An adjacent fragment refers to the 3′ end of the extension oligonucleotide being often 1 nucleotide from the 5′ end of the polymorphic site, and sometimes 2, 3, 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, and the number and/or type of nucleotides that are added to the extension oligonucleotide determine whether the polymorphic variant is present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. Oligonucleotide extension methods using mass spectrometry are described, for example, in U.S. Pat. Nos. 5,547,835; 5,605,798; 5,691,141; 5,849,542; 5,869,242; 5,928,906; 6,043,031; and 6,194,144. Multiple extension oligonucleotides may be utilized in one reaction, which is referred to as multiplexing.

Genetic mutations can be identified by hybridizing a sample and control nucleic acids, e.g., DNA or RNA, to high density arrays containing hundreds or thousands of oligonucleotides probes (Cronin M T et al., Hum Mutat 7(3):244-55; Kozal M J et al., 1996, Nat Med 2(7):753-9). For example, genetic mutations can be identified in two-dimensional arrays containing light-generated DNA probes as described in Cronin et al., (supra). Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Specific mutations can also be determined through direct sequencing of one or both strands of DNA using dideoxy nucleotide chain termination chemistry, electrophoresis through a semi-solid matrix and fluorescent or radioactive chain length detection techniques. Further mutation detection techniques may involve differential susceptibility of the polymorphic double strand to restriction endonuclease digestion, or altered electrophoretic gel mobility of single or double stranded gene fragments containing one polymorphic form. Other techniques to detect specific DNA polymorphisms or mutation may involve evaluation of the structural characteristics at the site of polymorphism using nuclear magnetic resonance or x-ray diffraction techniques.

An apparatus for detecting a nucleotide in a nucleic acid sequence is also provided. The apparatus comprises a substrate, such as a glass slide, and at least one oligonucleotide bound to the substrate. The oligonucleotide comprising a contiguous nucleic acid sequence complementary to CTGGACATTTTATATAGTGTGGGCTG[C/T]AACTTAAGTTTCACCGGGTGTGTCT (SEQ ID NO:1) and containing position 27 of the sequence, complementary to AGAAACCAGTTCAAAGCCTCCTGCAA[C/T]CCCCTAAAGTAAACAGAGACCAATA (SEQ ID NO:2) and containing position 27 of the sequence, or complementary to SEQ ID NO. 2 and containing position 27 of the sequence. In most cases, a second oligonucleotide will be bound to the substrate which corresponds to the oligonucleotide not already bound to the substrate. Preferably, the substrate will contain at least an oligonucleotide comprising a contiguous nucleic acid sequence complementary to SEQ ID NO. 1 and containing position 27 of the sequence and an oligonucleotide comprising a contiguous nucleic acid sequence complementary to SEQ ID NO. 2 and containing position 27 of the sequence. In another embodiment, the substrate will contain alone or in combination with SEQ ID NOs: 1 and 2, at least an oligonucleotide comprising a contiguous nucleic acid sequence complementary to ATTTGGAAAATGGATATAATCAAAAT[C/T]ATGGAAGAAAGTTTGTACAGGGTAA (SEQ ID NO. 3) and containing position 27 of the sequence.

Although the length of the oligonucleotides for use with the apparatus can be chosen in part based on the overall characteristics of the oligonucleotides on the substrate, a preferred range of lengths are between 25-mer and 60-mer.

A microarray can be utilized for determining whether the polymorphism is present or absent in a nucleic acid sample. A microarray may include any oligonucleotides described hereinabove, and methods for making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and the oligonucleotides may be linked to this solid support by covalent bonds or by non-covalent interactions. The oligonucleotides may also be linked to the solid support directly or by a spacer molecule. A microarray may comprise one or more oligonucleotides complementary to a polymorphism.

Unless otherwise specified, all references cited are incorporated herein.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein set forth, and as follows in the scope of the appended claims.

### EXAMPLES

### Subject Sample

Samples and corresponding genetic and supplement profiles came from the AREDS study. The study procedures have been reported elsewhere (see AREDS report no. 8, Arch Ophthalmol 119:1417-36, 2001).

Subjects used for the present study were classified based on the category of AMD in his or her best eye. Subjects chosen for observation had AREDS category 1 or 2. AREDS category 1 is considered a “normal” eye without any manifestations of age-related macular degeneration. AREDS category 2 is characterized by drusen measuring less than 125 μM and no retinal morphological evidence for choroidal neovascularization or geographic atrophy. The end point of observation was AREDS category 3 disease which is characterized as intermediate AMD, having 1 large drusen (>125 μm), extensive intermediate drusen, or geographic atrophy not involving the center of the macula.

Subjects with AREDS category 2 in at least one eye at base line were randomly prescribed oral tablets of placebo, antioxidants, zinc or antioxidants plus zinc. Individuals with normal eyes (AREDS category 1 in both eyes) were randomized only between placebo treatment and anti-oxidants. Antioxidants used in this study included daily doses of 15 mg of β-carotene, 500 mg of vitamin C, and 400 IU of vitamin E. Zinc supplements included 80 mg as zinc oxide and copper. Copper was included to prevent zinc-induced copper-deficiency anemia.

Subjects were examined every 6 months, and stereoscopic fundus photographs were obtained routinely from all eyes at baseline, at the 2-year follow-up visit, and every year thereafter. The average duration of treatment was 6.3 years.

The frequency of subject repeat retinal phenotyping allowed an accurate determination of progression from any category Individuals were considered to have progressed if a higher category of disease was documented compared to the preceding visit. For instance, individuals with an eye having AREDS category 1 phenotype (normal eyes) were considered to have progressed if the affected eye was shown to have category 2 or greater at a subsequent visit. Those with an eye with category 2 phenotype on any visit were considered to have progressed if the affected eye was shown to have category 3 or greater at a subsequent visit.

### Genotyping

Based on the genotype of the subject at rs3766405 and rs412852, the individual was categorized according to Table 1 as having a low risk, medium risk and high risk at the CFH locus. Based on the ARMS2 SNP.10490924 subjects were categorized as low (GG), and high risk (GT or TT).

An overall genetic risk score was generated based on the combination of risk alleles at these 2 loci as follows:

No: No AMD risk alleles at the CFH or ARMS2 loci.
Low: CFH low, ARMS2 High. OR CFH medium, ARMS2 low
Medium: CFH medium, ARMS2 High, CFH high, ARMS2 low,
High: CFH High, ARMS2 high.

### Statistical Analysis

The statistical analysis was performed using affected eyes as the basic unit of observation. All eyes observed had AREDS category 1 or 2 phenotypes. The selection of individual eyes as a unit of observation was done to allow evaluation of the effect of the status of the “other eye” on the observed gene-treatment effects. Eyes (n=2472) were divided into two groups: The first were those with AREDS category 1 phenotype with the other eye also being AREDS category 1. These individuals were considered to have 2 normal eyes. The second group had either AREDS category 1 disease or AREDS category 2 disease in the index eye and AREDS category 1 disease or AREDS category 2 disease in the other eye. The first group is a subset of the second group. Both groups were observed for progression from category 1 or 2 (as the case may be) to AREDS category 3. Patients who were noted to have progressed to AREDS category 3 phenotype were coded as a “case” irrespective of eye phenotype on subsequent visits. Based on genotyping data and information from the AREDS study data base, each case was classified according to 2 parameters: Treatment category (1, 2, 3 or 4) and genetic risk (the number of risk alleles at the CFH and ARMS2 loci) based on their genetic profile at the CFH SNPs rs3766405 and rs412852 and the ARMS2 SNP rs10490924. Patient groups were compared a cox regression survival analysis with a random-effect (so-called “frailty”) term to account for within-subject correlation. The p-values comparing hazard ratios across treatment groups and within a given genetic risk and eye-groups were obtained from the Cox model that included main effects for treatment and genetic group, interaction between these two effects, and was adjusted for age, gender and baseline AREDS category of the subject. The following chart shows the number of patients in each category. Categorical data of AMD risk factor occurring in different genetic risk groups were compared using a chi square analysis.

As shown in Table 1A and 1B, individuals receiving antioxidants or placebo did not differ with respect to genetic risk category. There was no difference in the distribution of risk alleles at CFH or ARMS2 for individuals in any of the 4 treatment groups. Table 1B shows distribution of genotypes among the eye phenotype groups. Individuals in phenotype group 1:1 were randomized only to antioxidants or placebo.

TABLE 1A

| Index eye | Other eye | Treatment distribution | | | | | | | |
|---|---|---|---|---|---|---|---|---|---|
| phenotype | phenotype | Placebo | | Antioxidants | | Zinc | | AREDSF | |
| 1 | 1 | 284 | | 270 | | 0 | | 0 | |
| 1 | 1-4 | 382 | | 387 | | 114 | | 115 | |
| 2 | 1 | 66 | | 81 | | 90 | | 79 | |
| 2 | 1-4 | 169 | | 198 | | 198 | | 188 | |
| Demographics | | | | | | | | | |
| Smoking (ever) p = 0.685 | | 230 | (47.4) | 240 | (47.6) | 115 | (51.8) | 105 | (46.9) |
| Body Mass Index p = 0.643 | | 27.3 | (4.4) | 27 | (4.8) | 27.4 | (4.5) | 27.3 | (4.6) |
| | | 268 | (55.3) | 271 | (53.8) | 133 | (59.9) | 132 | (58.9) |
| Sex (F) | | 217 | (44.7) | 233 | (46.2) | 89 | (40.1) | 92 | (41.1) |
| Sex (M) p = 0.35 | | | | | | | | | |
| Age (mean and SD) p = 0.109 | | 67.9 | (4.6) | 67.8 | (4.6) | 68.7 | (4.7) | 68.1 | (4.7) |
| Genetics (risk allele number) | | | | | | | | | |
| CFH p = | 0 | 144 | (29.7) | 148 | (29.4) | 72 | (32.4) | 59 | (26.3) |
| (0.46) | 1 | 247 | (50.9) | 273 | (54.2) | 104 | (46.8) | 125 | (55.8) |
| | 2 | 94 | (19.4) | 83 | (16.5) | 46 | (20.7) | 40 | (17.9) |
| ARMS2 | 0 | 287 | (59.2) | 318 | (63.1) | 123 | (55.4) | 118 | (52.7) |
| (p = 0.13) | 1 | 173 | (35.7) | 167 | (33.1) | 89 | (40.1) | 91 | (40.6) |
| | 2 | 25 | (5.2) | 19 | (3.8) | 10 | (4.5) | 15 | (6.7) |

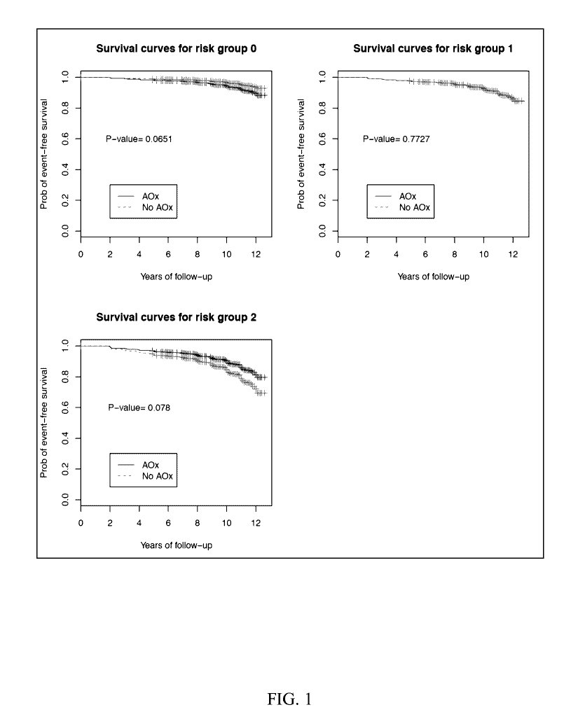

FIG. 1 and Table 2 show that individuals with normal eyes (AREDS category 1) without CFH risk alleles experience accelerated development of dry intermediate AMD if treated with an antioxidant formulation that includes a carotenoid. The other eye can be any stage of AMD including “normal” and the deleterious effect of antioxidants is still observed. Table 2 shows that there is a marginally statistical interaction between antioxidant treatment and the absence of CFH risk alleles with an interaction HR of 1.68 and a p value of 0.06. The overall p-value for significance of interaction between number of CFH alleles and antioxidant treatment is 0.022 and considered statistically significant.

TABLE 2

| | Eyes | Subject |
|---|---|---|
| Cases Counts: | 1552 | 998 |
| Phenotype (index: other) | Category 1: Category 1-4 | |
| CFH Allele | Progression AOx vs Placebo (Category 1−>3) | |
| Number | HR (10 year) | p value |
| 0 | 1.676 | 0.065 |
| 1 | 1.323 | 0.154 |
| 2 | 0.65 | 0.078 |

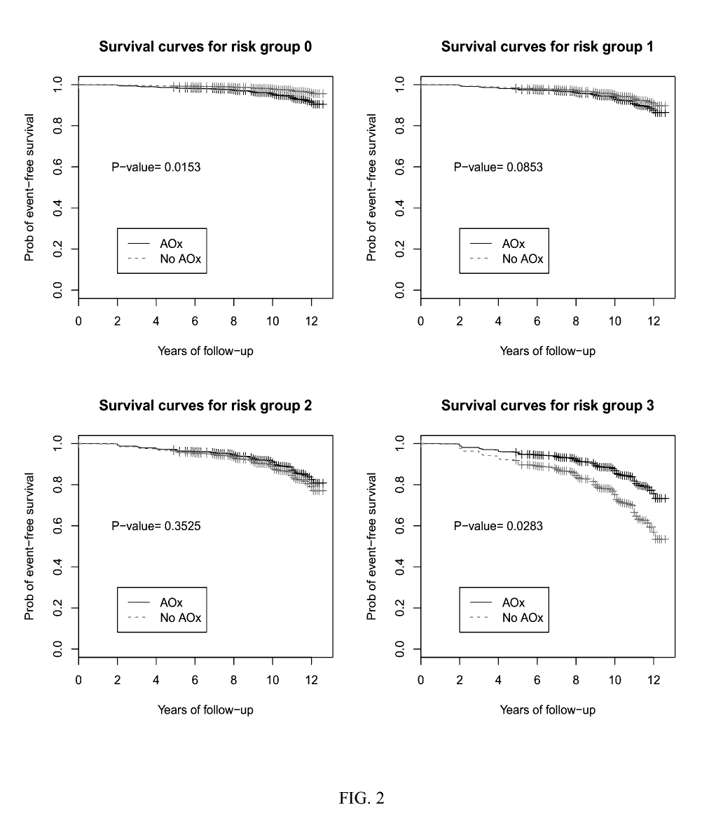

FIG. 2 and Table 3 show that individuals with normal eyes (AREDS category 1) with one CFH or ARMS2 risk allele experience accelerated development of dry intermediate AMD if treated with an anatioxidant formulation that includes a carotenoid when compared to placebo-treated individuals. In this data set the other eye can have any stage of AMD including “normal”. Specifically, if the individual has none, CFH or ARMS2 risk allele the hazard ratio for progression to dry intermediate AMD is 2.21, and individuals with 1 CFH or ARMS risk allele have hazard ratio of 1.38. In contrast, those that have 3 CFH and ARMS2 risk alleles experience reduced progression to dry intermediate AMD if treated with an antioxidant formulation that includes a carotenoid compared to placebo-treated with an hazard ratio of approximately 0.536 with a highly significant p value of 0.028. Table 3 shows that there is a statistically significant interaction between antioxidant treatment and the number of CFH or ARMS2 risk alleles for 0, 1, and 3 CFH and ARMS2 risk alleles and an overall interaction p value is 0.0057 which is highly statistically significant. This indicates that genetic stratification can distinguish benefit from harm associated with antioxidant and carotenoid therapy.

TABLE 3

| | Eyes | Subjects |
|---|---|---|
| Cases Counts: | 1552 | 998 |
| Phenotype (index: other) | Category 1: Category 1-4 | |
| CFH and ARMS2 Risk | Progression AOx vs Placebo (Category 1−>3) | |
| Allele Number | HR (10 year) | p value |
| 0 | 2.21 | 0.015 |
| 1 | 1.38 | 0.085 |
| 2 | 0.86 | 0.353 |
| 3 | 0.536 | 0.028 |

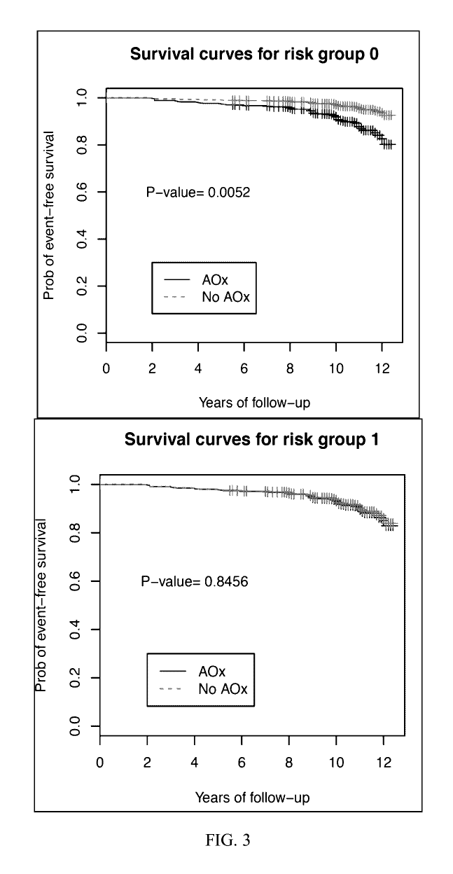

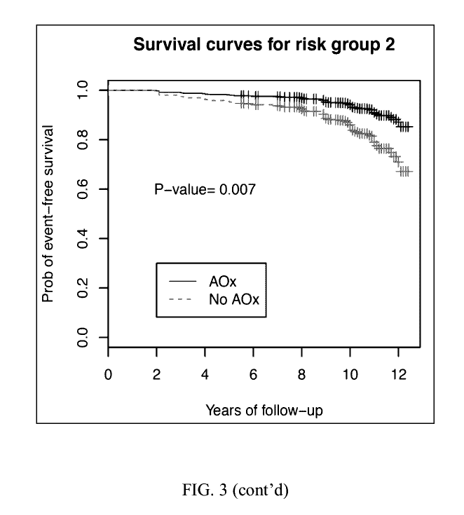

FIG. 3 and Table 4 show that individuals with normal eyes without CFH risk alleles experience accelerated development of dry intermediate AMD if treated with an antioxidant formulation that includes a carotenoid. The data presented here is for individuals having a normal other eye. Specifically, if the individual has no CFH risk alleles the hazard ratio for progression to dry intermediate AMD is 2.85, and for individuals with 1 CFH risk allele, the hazard ratio is approximately 1.75. In contrast, those that having 2 risk alleles experience reduced progression to dry intermediate AMD if treated with an antioxidant formulation that includes a carotenoid compared to placebo-treated with an hazard ratio of 0.405, which is statistically significant at the 0.01 level. Table 4 shows that the number of AMD risk alleles at the CFH locus interacts statistically with antioxidant administration with a combined interaction p=0.0006. This demonstrates that the deleterious effect of antioxidants is more pronounced in individuals with 2 normal eyes at the initiation of antioxidant and carotenoid supplementation than if the other eye is affected with early AMD (compare Table 2 and Table 4). This is relevant for the use of antioxidants with carotenoids in people with bilateral normal eyes—some of whom will experience accelerated progression to dry intermediate AMD based on the number of CFH risk alleles if treated with an antioxidant with a carotenoid.

TABLE 4

| | Eyes | Subjects |
|---|---|---|
| Cases Counts: | 1108 | 554 |
| Phenotype (index: other) | Category 1: Category 1 | |
| CFH Allele Number | Progression AOx vs Placebo (Category 1−>3) | |
| (Risk Group) | HR (10 year) | p value |
| 0 | 2.846 | 0.004 |
| 1 | 1.748 | 0.029 |
| 2 | 0.405 | 0.01 |

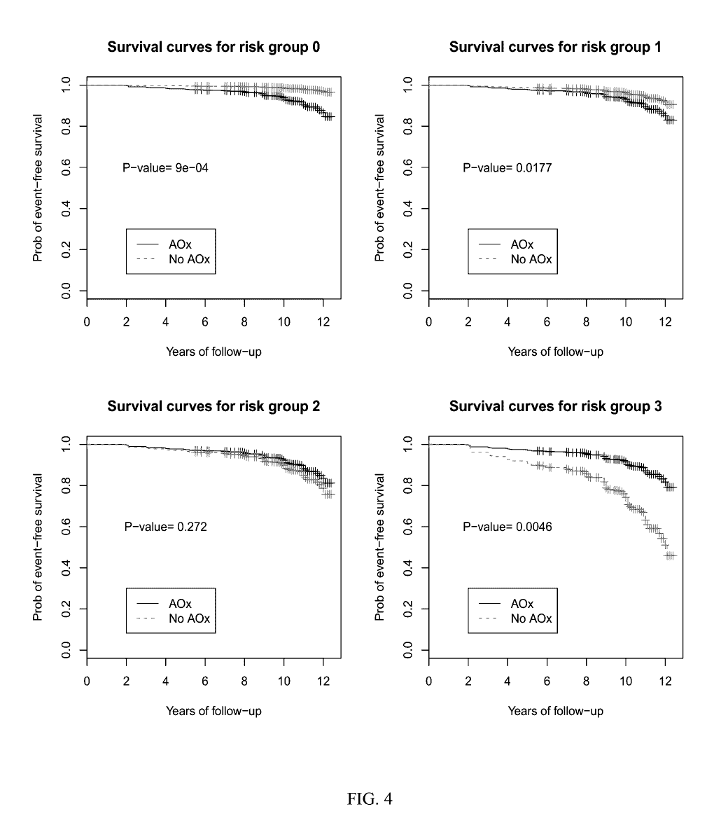

FIG. 4 and Table 5 show that individuals with normal eyes with only 1 CFH or ARMS2 risk allele experience accelerated development of dry intermediate AMD if treated with an antioxidant formulation that includes a carotenoid. The data presented here is for individuals having a normal other eye. Specifically, if the individual has none, or 1 CFH or ARMS2 risk allele the hazard ratio for progression to dry intermediate AMD is 4.33. In contrast, those that have 3 CFH and ARMS2 risk alleles experience reduced progression to dry intermediate AMD if treated with an antioxidant formulation that includes a carotenoid compared to placebo-treated with an hazard ratio of approximately 0.331, which is significant statistically at the 0.005 level. Table 5 shows that the number of AMD risk alleles at the CFH and ARMS2 risk loci interact statistically with antioxidant administration with an interaction p value of, p=0.00016. This demonstrates that the deleterious effect of antioxidants is more pronounced in individuals with 2 normal eyes at the time of initiation of antioxidant and carotenoid supplementation than if the other eye is affected with early AMD (comparing Table 3 and Table 5). This is relevant for the use of antioxidants with carotenoids in people with bilateral normal eyes—some of whom will experience accelerated progression to dry intermediate AMD based on the number of CFH risk alleles if treated with an antioxidant with a carotenoid.

TABLE 5

| | Eyes | Subjects |
|---|---|---|
| Cases Counts: | 1108 | 554 |
| Phenotype (index: other) | Category 1: Category 1 | |
| CFH and ARMS2 Risk | Progression AOx vs Placebo (Category 1−>3) | |
| Allele Number | HR (10 year) | p value |
| 0 | 4.333 | <0.001 |
| 1 | 2.838 | 0.018 |
| 2 | 0.779 | 0.272 |
| 3 | 0.331 | 0.005 |

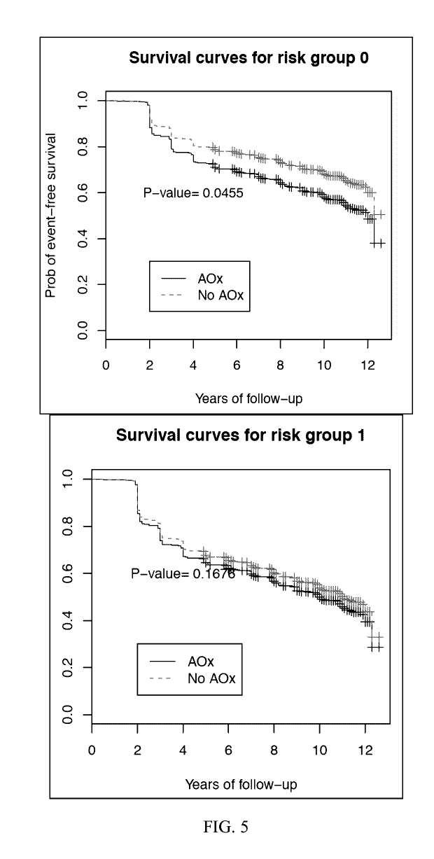

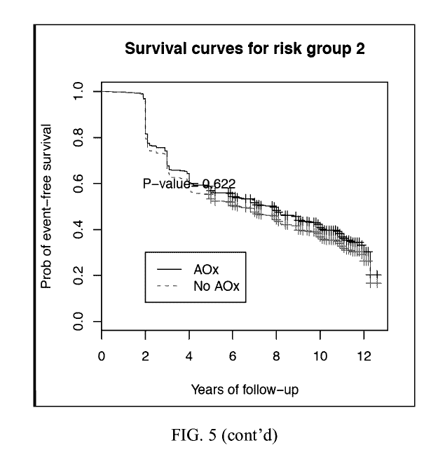

FIG. 5 and Table 6 show that individuals with early dry AMD (AREDS category 2) with no CFH risk alleles experience accelerated development of dry intermediate AMD if treated with an antioxidant formulation that includes a carotenoid. Data is presented here that shows the differential effects of antioxidants and a carotenoid in patients with the index eye having AREDS category 2 disease and the other eye having any class of AMD (AREDS category 1-4). In the absence of any CFH risk alleles the hazard ratio for progression to dry intermediate AMD is 1.45, and with 1 CFH risk allele the hazard ratio is still elevated at 1.30. Table 6 shows that the absence of 2 CFH risk alleles and the administration of antioxidants with a carotenoid leads to elevated risk of progression to dry intermediate AMD while individuals with 2 CFH risk alleles experience no such deleterious effect of antioxidant treatment. This indicates that genetic stratification on the basis of the CFH gene alone can distinguish benefit from harm associated with antioxidant and carotenoid therapy in eyes with early AMD who may or may not progressed to intermediate dry AMD.

TABLE 6

| | Eyes | Subjects |
|---|---|---|
| Cases Counts: | 920 | 753 |
| Phenotype (index: other) | Category 2: Category 1-4 | |
| CFH Allele | Progression AOx vs Placebo (Category 2−>3) | |
| Number | HR (10 year) | p value |
| 0 | 1.445 | 0.032 |
| 1 | 1.295 | 0.032 |
| 2 | 0.932 | 0.671 |

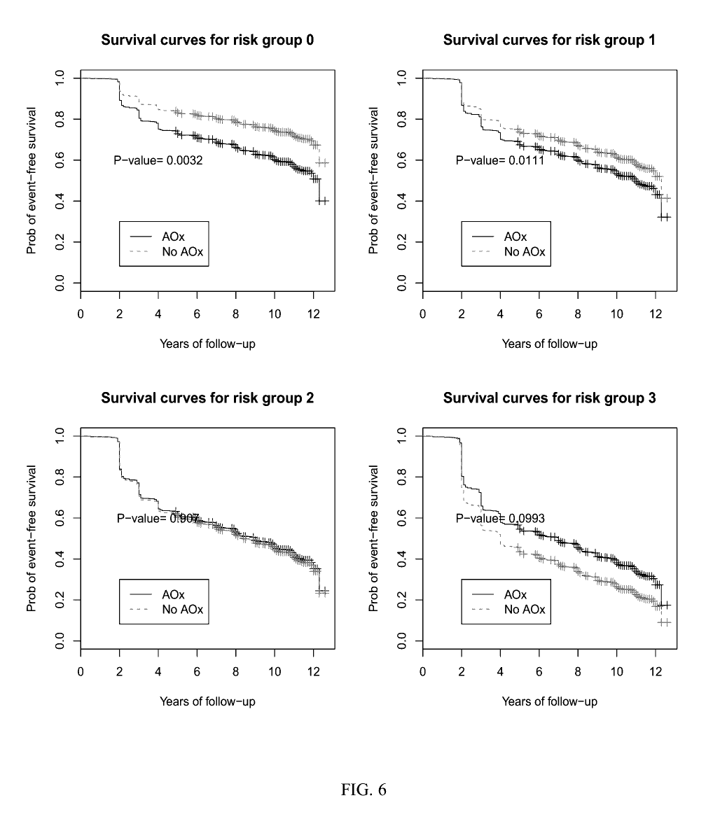

FIG. 6 and Table 7 show that eyes with AREDS category 2 disease (drusen<125 μM) from patients in which the other eye is normal or has any category of AMD (AREDS category 1-4) experience accelerated development of dry intermediate AMD if treated with an antioxidant formulation containing a carotinoid if they have 0 or 1 CFH or ARMS2 risk alleles and have reduced progression to intermediate dry AMD if they have 3 CFH and ARMS2 risk alleles. Specifically, if there are no CFH or ARMS2 risk alleles the hazard ratio for progression to dry intermediate AMD is 1.80 and if the sum of CFH and ARMS2 risk alleles is 1, the hazard ratio is approximately 1.33. In contrast, those that have 3 CFH and ARMS2 risk alleles experience reduced progression to dry intermediate AMD if treated with an antioxidant formulation that includes a carotenoid relative to placebo-treatment with an hazard ratio of approximately 0.731. Table 7 shows that the number of AMD risk alleles at the CFH and ARMS2 loci interact statistically in this population with the administration of antioxidants resulting in a combined p=0.0071. This indicates that genetic stratification on the bases of the number of CFH or ARMS2 risk alleles can distinguish benefit from harm associated with antioxidant and carotenoid therapy in individuals with early AMD (AREDS category 2) in one eye and any degree of AMD in the other eye.

TABLE 7

| | Eyes | Subjects |
|---|---|---|
| Cases Counts: | 920 | 753 |
| Phenotype (index: other) | Category 2: Category 1-4 | |
| CFH and ARMS2 Risk | Progression AOx vs Placebo (Category 2−>3) | |
| Allele Number | HR (10 year) | p value |
| 0 | 1.803 | 0.003 |
| 1 | 1.334 | 0.011 |
| 2 | 0.987 | 0.907 |
| 3 | 0.731 | 0.099 |

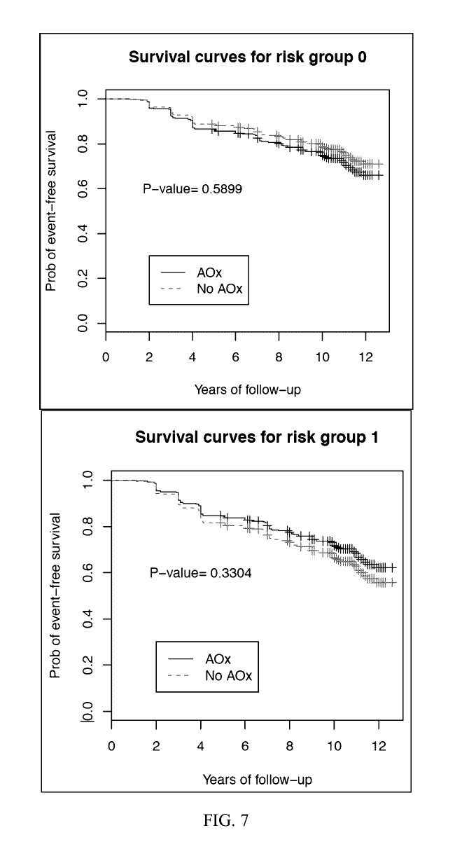

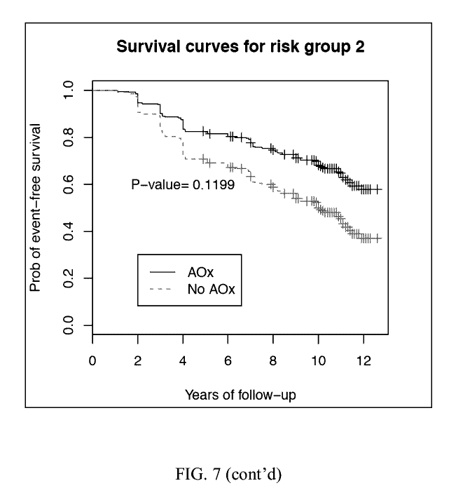

FIG. 7 and Table 8 show that an eye with AREDS classification 2 disease (drusen<125 μM) in patients in which the other eye is normal (AREDS category 1) experience accelerated development of dry intermediate AMD if treated with an antioxidant formulation if they have no CFH risk alleles. Specifically, if the individual has 0 CFH risk allele, the hazard ratio for progression to dry intermediate AMD is 1.36. In contrast, those that have 2 CFH risk alleles experience reduced progression to dry intermediate AMD if treated with an antioxidant formulation that includes a carotenoid relative to placebo-treatment with an hazard ratio of 0.57. Table 8 shows that the absence of CFH risk alleles and antioxidant therapy interact statistically with a combined p=0.15. This indicates that genetic stratification on the basis of CFH risk alleles can distinguish benefit from harm associated with antioxidant and carotenoid therapy in individuals with 2 normal eyes.

TABLE 8

| | Eyes | Subjects |
|---|---|---|
| Cases Counts: | 316 | 316 |
| Phenotype (index: other) | Category 2: Category 1 | |
| CFH Allele | Progression AOx vs Placebo (Category 1−>3) | |
| Number | HR (10 year) | p value |
| 0 | 1.357 | 0.391 |
| 1 | 1.093 | 0.723 |
| 2 | 0.57 | 0.14 |

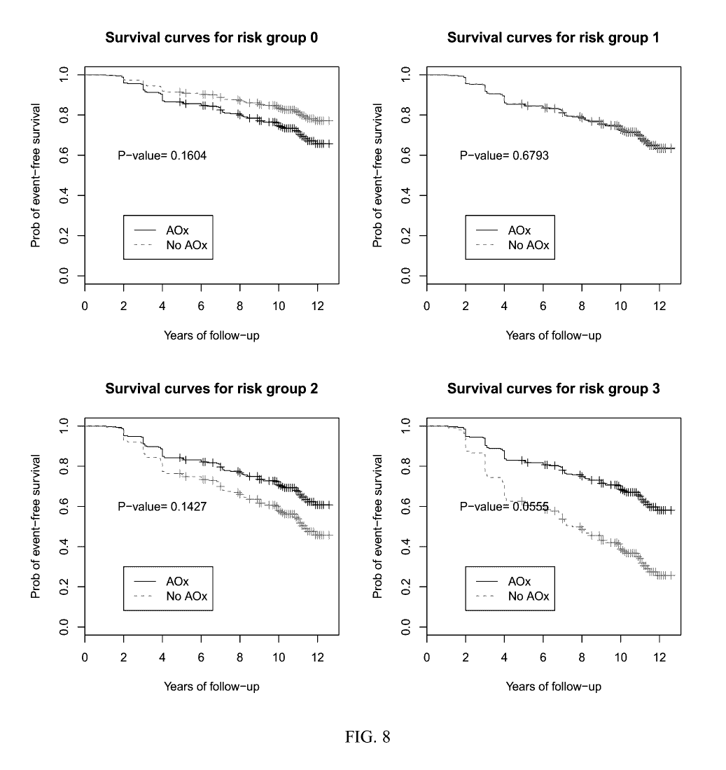

FIG. 8 and Table 9 show that eyes with AREDS category 2 disease (drusen<125 μM) from patients in which the other eye is normal (AREDS category 1) experience accelerated development of dry intermediate AMD if treated with an antioxidant formulation containing a carotinoid if they have 0 CFH or ARMS2 risk alleles and have reduced progression to intermediate dry AMD if they have 3 CFH and ARMS2 risk alleles. Specifically, if there are no CFH or ARMS2 risk alleles, the hazard ratio for progression to dry intermediate AMD is 1.76. In contrast, those that have 3 CFH and ARMS2 risk alleles experience reduced progression to dry intermediate AMD if treated with an antioxidant formulation that includes a carotenoid relative to placebo-treatment with an hazard ratio of approximately 0.429 with a statistical p value of 0.055. Table 7 shows that the number of AMD risk alleles at the CFH and ARMS2 loci interact statistically in this population with the administration of antioxidants resulting in a combined, p=0.05. This indicates that genetic stratification on the bases of the number of CFH or ARMS2 risk alleles can distinguish benefit from harm associated with antioxidant and carotenoid therapy in individuals with early AMD (AREDS category 2) in the index eye and no AMD in the other eye (AREDS category 1).

TABLE 9

| | Eyes | Subjects |
|---|---|---|
| Cases Counts: | 316 | 316 |
| Phenotype (index: other) | Category 2: Category 1 | |
| CFH and ARMS2 Risk | Progression AOx vs Placebo (Category 2−>3) | |
| Allele Number | HR (10 year) | p value |
| 0 | 1.764 | 0.16 |
| 1 | 1.102 | 0.679 |
| 2 | 0.688 | 0.143 |
| 3 | 0.429 | 0.055 |

Table 10 shows the specificity of the interaction between low risk CFH genotypes and ARMS2 genotypes with antioxidant and carotenoid treatment. The progression to intermediate dry AMD (AREDS category 3) from normal eyes or eyes with early dry AMD (AREDS category 2 disease), is not affected by supplementation with zinc therapy. As shown in this table, the statistical interaction term between zinc therapy and the number of AMD risk alleles is not significant.

TABLE 10

| | Eyes | Subjects |
|---|---|---|
| Cases Counts: | 2472 | 1435 |
| Phenotype (index: other) | Category 1 or2: Category 1 | |
| CFH and ARMS2 Risk | Progression AOx vs Placebo (Category 2−>3) | |
| Allele Number | HR (10 year) | p value |
| 0 | 0.807 | 0.222 |
| 1 | 0.835 | 0.081 |
| 2 | 0.865 | 0.132 |
| 3 | 0.895 | 0.500 |

TABLE 1B

| | | CFH or ARMS2 | | | | CFH allele | | |
|---|---|---|---|---|---|---|---|---|
| Index eye | Other eye | allele number | | | | number | | |
| phenotype | phenotype | 0 | 1 | 2 | 3 or 4 | 0 | 1 | 2 |
| | | Antioxidant Treated | | | | | | |
| 1 | 1 | 49 | 128 | 83 | 10 | 76 | 158 | 36 |
| 1 | 1-4 | 103 | 214 | 162 | 23 | 151 | 276 | 75 |
| 2 | 1 | 39 | 60 | 49 | 12 | 54 | 79 | 27 |
| 2 | 1-4 | 73 | 147 | 122 | 44 | 110 | 201 | 75 |
| | | No Antioxidants | | | | | | |
| 1 | 1 | 60 | 114 | 92 | 18 | 92 | 138 | 54 |
| 1 | 1-4 | 90 | 218 | 158 | 30 | 157 | 250 | 89 |
| 2 | 1 | 23 | 73 | 51 | 9 | 49 | 82 | 25 |
| 2 | 1-4 | 52 | 155 | 129 | 31 | 108 | 183 | 76 |

1. A method for determining a supplement composition for preventing development of dry intermediate AMD (AREDS category 3) in a subject, said method comprising the steps of:
(a) classifying the subject as having normal eyes (AREDS category 1) or mild dry age-related macular degeneration (AMD)(AREDS category 2);
(b) determining an AMD risk allele profile based on the genetic profile of the subject at the complement factor H (CFH) and ARMS2 loci;
(c) prescribing a supplement based on the AMD risk allele profile at the CFH and ARMS2 loci, wherein 1) a subject classified as having normal eyes or mild dry AMD having low risk of developing dry intermediate AMD at CFH loci and low risk at ARMS2 loci or medium risk at CFH loci and low risk at ARMS2 loci or low risk at CFH loci and medium risk at ARMS2 locus is prescribed a supplement without antioxidants; and 2) a subject classified as having normal eyes or mild dry AMD having high risk of developing intermediate dry AMD at CFH loci and medium risk at ARMS2 locus risk alleles at the CFH and ARMS2 loci is prescribed a supplement with antioxidants.

2. The method of claim 1, wherein the subject's risk of developing dry intermediate AMD (AREDS category 3) is determined by analysing the single nucleotide polymorphisms: rs3766405 and rs412852 in the CFH gene and the rs10490924 SNP in the ARMS2 gene.

3. The method of claim 1, wherein the subject's risk of developing dry intermediate AMD (AREDS category 3) is determined by analysing the single nucleotide polymorphisms: rs1048663, rs3766405, rs412852, rs11582939 and/or rs1280514 in the CFH gene.

4. The method of claim 1, wherein the subject's risk of developing dry intermediate AMD (AREDS category 3) is determined by analysing the single nucleotide polymorphism rs1061170.

5. The method of claim 1, wherein the supplement with antioxidants comprises vitamin C, vitamin E, beta carotene, other carotinoids or combinations of any of these.

6. The method of claim 5, wherein the other carotinoids are lutein and zeazanthin.

7. The method of claim 1, wherein the subject is considered at high risk of developing dry intermediate AMD (AREDS category 3) when the subject is homozygous for the C allele at rs3766405 and is homozygous for the C allele at rs412852.

8. The method of claim 1 wherein the subject is considered at low risk of developing dry intermediate AMD (AREDS category 3) when the subject is heterozygous for the C allele at rs3766405 and is homozygous for the T allele at rs412852; or homozygous for the T allele at rs3766405 and homozygous for the T allele at rs412852.

9. The method of claim 3, wherein the subject is considered at medium risk of developing dry intermediate AMD (AREDS category 3) when the subject is homozygous for the C allele at rs3766405 and is homozygous for the T allele at rs412852, heterozygous for the C allele at rs3766405 and is homozygous for the C allele at rs412852, heterozygous for the C allele at rs3766405 and is heterozygous for the C allele at rs412852, homozygous for the T allele at rs3766405 and is homozygous for the C allele at rs412852, homozygous for the T allele at rs3766405 and is heterozygous for the C allele at rs412852 or homozygous for the C allele at rs3766405 and is heterozygous for the C allele at rs412852.

10. The method of claim 4, wherein the subject is considered at high risk of developing dry intermediate AMD (AREDS category 3) when the subject is homozygous for the C allele at rs1061170.

11. The method of claim 4, wherein the subject is considered at medium risk of developing dry intermediate AMD (AREDS category 3) when the subject is heterozygous for the C allele at rs1061170 or when subject is homozygous for the T allele in rs1061170.

12. The method of claim 1, wherein the subject's genetic profile is detected by hybridization, chemical cleavage, direct DNA sequencing, use of restriction enzymes or Southern blotting.


SEQUENCE LISTING (3 sequences)

SEQ ID NO: 1 - protein, 52 aa, Homo sapiens
  misc_feature (27)..(27): Xaa can be Cys or Thr
CTGGACATTTTATATAGTGTGGGCTGXAACTTAAGTTTCACCGGGTGTGTCT

SEQ ID NO: 2 - protein, 52 aa, Homo sapiens
  misc_feature (27)..(27): Xaa can be Cys or Thr
AGAAACCAGTTCAAAGCCTCCTGCAAXCCCCTAAAGTAAACAGAGACCAATA

SEQ ID NO: 3 - protein, 52 aa, Homo sapiens
  misc_feature (27)..(27): Xaa can be Cys or Thr
ATTTGGAAAATGGATATAATCAAAATXATGGAAGAAAGTTTGTACAGGGTAA